(19) United States
(12) Patent Application Publication
(10) Pub. No.: US 2015/0164760 A1
(43) Pub. Date: Jun. 18, 2015

(54) HAIR COLOURING COMPOSITION COMPRISING GOSSYPETIN

(71) Applicant: Conopco, Inc., d/b/a UNILEVER, Englewood Cliffs, NJ (US)

(72) Inventors: Ranjit Kaur Bhogal, Huntingdon (GB); John Casey, Yelden (GB); Shovan Ganguli, Bangalore (IN); Karl John Hunter, Bedford (GB); Jean Hypolites Koek, Schiedam (NL); Sally Pamela Redfern, Sharnbrook (GB)

(21) Appl. No.: 14/407,477

(22) PCT Filed: Jun. 19, 2013
(86) PCT No.: PCT/EP2013/062699

§ 371 Date: Dec. 12, 2014

(30) Foreign Application Priority Data
Jun. 21, 2012 (IN) 1775/MUM/2012
Aug. 15, 2012 (EP) 12180570.9

(51) Int. Cl. A61K 8/49; A61K 8/66; A61K 8/22; A61Q 5/10

(52) U.S. Cl. CPC A61K 8/498; A61Q 5/10; A61K 8/66; A61K 8/22

(57) ABSTRACT

Hair colourant formulations fall into three main categories designated permanent, semi-permanent and temporary. Permanent hair colourant formulations are oxidative dye systems and generally contain paraphenylene diamine (PPD) and resorcinol, both of which have been shown to cause sensitisation and mutagenicity. Furthermore, severe oxidising conditions are required which in themselves cause skin irritation and sensitization as well as hair fibre damage. The inventive composition addresses the aforementioned disadvantages by providing a hair colour composition comprising: (a) Gossypetin; and either (b1) A combination of a peroxidase with either a hydrogen peroxide generator or hydrogen peroxide, or (b2) a laccase; wherein the composition has a pH of 4.5 to 7.0, preferably less than or equal to 6.0.

A hair colour composition is disclosed, the composition comprising the flavonol gossypetin, and either a combination of a peroxidase with either a hydrogen peroxide generator or hydrogen peroxide, or a laccase, wherein the composition has a pH of 4.5 to 7.0, preferably less than or equal to 6.0. Optionally the hair colour composition includes metal ions, for example iron or copper, which are able to generate a range of colours for dyeing hair through coordination chemistry with gossypetin, or reaction products thereof with the enzyme.

Hair colourant formulations fall into three main categories designated permanent, semi-permanent and temporary. They vary in their degree of wash-fastness where permanent hair colourant formulations last 30-40 shampoo cycles (4-6 weeks) and temporary systems are rinsed out during the first wash. Permanent hair colourant formulations are oxidative dye systems and generally contain paraphenylene diamine (PPD) and resorcinol, both of which have been shown to cause sensitisation and mutagenicity. Furthermore the severe oxidising conditions required (hydrogen peroxide concentrations of 3% or more and a pH of 8.5 or higher for time periods of 20 minutes or more) in themselves can cause skin irritation and sensitization for some individuals as well as hair fibre damage. In addition, permanent hair colourant formulations also contain ammonia, used to swell the hair and lift the cuticle scales to allow penetration of dye precursors, which gives off a strong and unpleasant odour.

Takada et al. (Journal of Oleo Science, 52, 10, 557-563 (2003)) discloses a hair colouring composition comprising laccase and the flavan-3-ol catechin at pH 5.

WO 99/16893 (Instituut voor Agrotechnologisch Onderzoek (ATO-DLO)) discloses an enzymatic method for the synthesis of organic dyes, comprising bringing together an oxidative enzyme, a hydrogen acceptor and a hydrogen donor wherein at least the hydrogen acceptor or donor is a substrate. In particular, Example 3 discloses reactions between peroxidase, hydrogen peroxide and the flavonols myricetin or quercetin.

The invention addresses the aforementioned disadvantages by providing a hair colouring compositions which avoids the issues of safety and toxicity associated with the known synthetic oxidative systems, causes less damage by avoiding the use high levels of hydrogen peroxide, whilst maintaining colour fastness.

### SUMMARY OF THE INVENTION

In a first aspect of the invention, a hair colour composition is provided, the composition comprising:

(a) Gossypetin; and either
(b1) A combination of a peroxidase with either a hydrogen peroxide generator or hydrogen peroxide, or
(b2) a laccase;
wherein the composition has a pH of 4.5 to 7.0, preferably less than or equal to 6.0.

In a second aspect of the invention, a method of colouring hair fibres is provided, the method comprising the step of applying the hair colour composition of the first aspect of the invention to hair fibres.

### DETAILED DESCRIPTION OF THE INVENTION

As consumers will benefit from avoiding the issues around allergic skin reactions, safety and toxicity of current permanent hair colourant formulations, the hair colour composition may be applied more frequently, for example daily, in the form of, for example, a shampoo or conditioner thereby addressing the problem of grey hair or natural hair colour re-growth observed during the intervals between hair dyeing treatments. Furthermore by multiple consecutive applications of the hair colour composition, the consumer can also control the degree of colour desired.

The hair colour composition may comprise 0.001-10, preferably 0.1-5% w/w (+)-gossypetin.

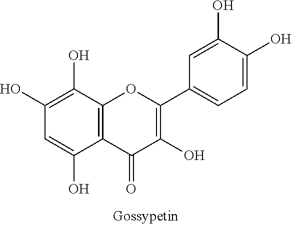 [SMILES: O=c1c(O)c(-c2ccc(O)c(O)c2)oc2c(O)c(O)cc(O)c12]

The peroxidase or laccase is selected from those which are suited to neutral or slightly acid pH, that is to say a pH of 4.5 to 7.0, preferably a pH of less than or equal to 6.0. The advantage of this is that this more closely conforms to the pH of the skin thereby leading to lower levels of skin irritation. The peroxidase is preferably a non-animal haem peroxidase from class II (fungi) or class III (plants and algae), most preferably obtained from the group consisting of Arabidopsis thaliana, horse radish, barley, peanut soybean, tobacco, and turnip (plants), Chlorophyta spirogyra (green algae), Arthromyces ramosus and Corprinus cinereus (fungi). Alternatively the hair colour composition may comprise a laccase originating from a fungal or a plant source such as those from the group consisting of the Aspergillus, Botrytis, Ceriporiopsis, Cerrena, Chaetomium, Coprinus, Coriolus, Neurospora, Panus, Phanerochaete, Pleurotus, Polyporus, Pycnoporus and Trametes genera, and Rhus vernicifera. In either case, the hair colour composition may comprise 0.0001-5, preferably 0.0001-1% w/w peroxidase or laccase.

When using a peroxidase, a hydrogen peroxide generator is preferred because it reduces the aforementioned problems associated with the use of large amounts of hydrogen peroxide whilst providing a constant supply of hydrogen peroxide. Preferably the hydrogen peroxide generator comprises a hydrogen peroxide generating oxidase, a substrate and oxygen. Alternatively a chemical system can be used to generate hydrogen peroxide such as one based on ascorbate and a transition metal ion, or anthraquinone monosulphate and glucose (T. Vuorinen, Carbohydrate Research, 127, 2, 319-325 (15 Apr. 1984)).

The hydrogen peroxide generating oxidase may be selected from the group consisting of (S)-2-hydroxy acid oxidase, D-galactose oxidase, glucose oxidase, coniferyl alcohol oxidase, glycolate oxidase, hexose oxidase, oxalate oxidase, amino acid oxidase and L-galactonolactone oxidase and the respective substrate is selected from the group consisting of (S)-2-hydroxy acid, D-galactose, glucose, coniferyl alcohol, α-hydroxy acids, D-glucose, oxalic acid, amino acid and L-galactono-1,4-lactone.

More preferably the hydrogen peroxide generator is selected from the group consisting of (S)-2-hydroxy acid with (S)-2-hydroxy acid oxidase, D-galactose with D-galactose oxidase, glucose with glucose oxidase, coniferyl alcohol with coniferyl alcohol oxidase, α-hydroxy acids with glycolate oxidase, D-glucose with hexose oxidase, oxalic acid with oxalate oxidase, amino acid oxidase with amino acid and L-galactono-1,4-lactone with L-galactonolactone oxidase, all in the presence of oxygen.

When using peroxidase, the hair colour composition may comprise 0.0001-3 preferably 0.001-1, most preferably 0.01-1% w/w hydrogen peroxide. However if a hydrogen peroxide generator is selected, then the hair colour composition may comprise 0.0001-5, preferably 0.001-1% w/w hydrogen peroxide generating oxidase, and 0.01-10, preferably 0.1-5% w/w substrate.

The hair colour composition of the first aspect may additionally comprise a metal ion suitable for coordinating to gossypetin, or the product of the reaction of gossypetin in the presence of either the peroxidase and hydrogen peroxide, or laccase. The metal ion may be selected from the group consisting of iron (II), iron (III), copper (I), copper (II), and aluminium (III). The hair colour composition may comprise 0.0001-2, preferably 0.001-0.1% w/w metal ion.

### Example

### Materials

Myceliphthora thermophila laccase (51003) was obtained from Novozymes NS (Denmark)

Soy bean peroxidase (SBP) was obtained from Bio-Research Products Incorporated (SBP#510)

3% aqueous hydrogen peroxide (H2O2, 323381) was obtained from Sigma-Aldrich (UK)

(+)-Catechin hydrate (C1251) was obtained from Sigma-Aldrich (UK)

Gossypetin (1176) was obtained from Extrasynthese (France)

Myricetin (M6760) was obtained from Sigma-Aldrich (UK)

Quercetin dihydrate (171964) was obtained from Sigma-Aldrich (UK)

Natural White hair switches were obtained from International Hair Importers (New York, USA)

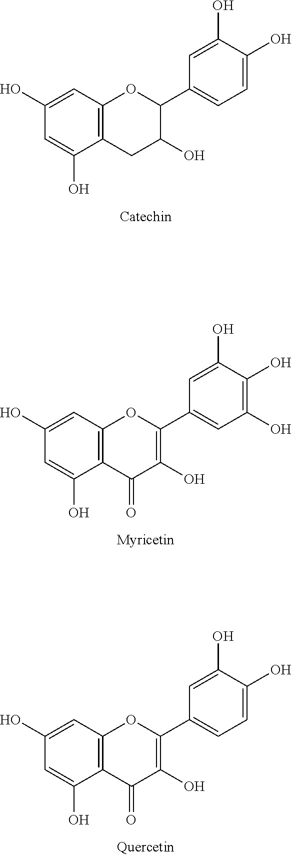 [SMILES: Oc1cc(O)c2c(c1)OC(c1ccc(O)c(O)c1)C(O)C2 ; O=c1c(O)c(-c2cc(O)c(O)c(O)c2)oc2cc(O)cc(O)c12 ; O=c1c(O)c(-c2ccc(O)c(O)c2)oc2cc(O)cc(O)c12]

### Method:

Two inch/0.25 g hair switches were washed with 1% aqueous sodium dodecyl sulphate (SDS), followed by 10% aqueous SDS solutions, rinsed and dried prior to use. Baseline L*a*b* colour measurements were made on all hair switches (6 per switch) prior to dyeing using a Konica Minolta Spectrophotometer CM-2600d. Hair dyeing was carried out in 15 ml Falcon tubes in a 10 ml final volume.

For SBP, hair switches were soaked in 8 ml of polyphenol in ethanol/Britton Robinson buffer (pH 6.0) for 10 minutes at room temperature. 1 ml of SBP in Britton Robinson buffer (pH 6.0) and 1 ml of 3% aqueous hydrogen peroxide were added, such that the final concentrations were:

1-5 mM polyphenol*
5-28% ethanol
10 U·ml⁻¹ SBP
0.3% hydrogen peroxide * Gossypetin (all 1 mM); myricetin (2 mM); catechin, quercetin (both 5 mM)

With respect to laccase, hair switches were soaked in 8 ml of polyphenol in ethanol/Britton Robinson buffer (pH 6.0) for 10 minutes at room temperature. 1 ml of laccase in Britton Robinson buffer (pH 6.0) and 1 ml of water were added, such that the final concentrations were:

1-5 mM polyphenol **
5-28% ethanol
10 U·ml⁻¹ laccase ** Gossypetin (1 mM); myricetin (2 mM); catechin, quercetin (both 5 mM)

After 10 minutes in the polyphenol/enzyme mixture, switches were rinsed in running tap water until the water ran clear. The hair switch was then dried with a hair dryer and L*a*b* colour measurements taken (6 measurements per switch). This process was repeated up to 4 times. Afterwards the hair switches were washed with 50 μl of shampoo, rinsed in water and dried. This was repeated 4 times. Shampooing was accomplished by fully wetting the switches in tap water and then applying 50 μL of shampoo was to each switch, lathering for 2 minutes before rinsing and drying. L*a*b* colour measurements were then taken to assess the amount of colour loss after each shampoo treatment.

ΔE* values were calculated according to the equation below:

ΔE=√(L*_{B}−L*_{D})²+(a*_{B}−a*_{D})²+(b*_{B}−b*_{D})²

Where B=background/baseline and D=dyed;
L*=lightness (where 0=black and 100=diffuse white);
a*=green/red (negative values indicate green and positive values indicate red); and
b*=blue/yellow (negative values indicate blue and positive values indicate yellow)

### Results:

The results are summarised below in Table 1. L*, a* and b* values were observed to remain substantially constant after each shampoo cycle.

TABLE 1
ΔE*, L*, a* and b* values after four treatments for enzymatically catalysed oxidation
of gossypetin, catechin, myricetin and quercetin on white hair switches (n = 6).

| | Four treatments | | | |
|---|---|---|---|---|
| | ΔE* | ΔL* | Δa* | Δb* |
| Laccase | | | | |
| Gossypetin | 40.16 ± 2.41 | −36.67 ± 1.87 | −7.45 ± 0.28 | −14.52 ± 2.01 |
| Catechin | 11.64 ± 1.87 | −5.55 ± 1.42 | 5.87 ± 0.98 | 8.32 ± 1.26 |
| Peroxidase | | | | |
| Gossypetin | 28.51 ± 3.12 | −25.79 ± 2.94 | −4.39 ± 0.73 | −11.28 ± 1.45 |
| Quercetin | 8.44 ± 2.00 | −3.55 ± 1.13 | −1.20 ± 0.18 | 7.48 ± 2.08 |
| Myricetin | 17.16 ± 1.45 | −13.48 ± 1.15 | 8.87 ± 0.64 | 5.81 ± 0.95 |

### CONCLUSION

Gossypetin in combination with either laccase or peroxidase/hydrogen peroxide provides a significantly darker shade (ΔL*) than any of the prior art combinations. Colour fastness after shampoo treatment was observed in all cases.

1. A hair colour composition comprising:
(a) Gossypetin; and either
(b1) A combination of a peroxidase with either a hydrogen peroxide generator or hydrogen peroxide, or
(b2) a laccase;
wherein the composition has a pH of 4.5 to 60.

2. A hair colour composition according to claim 1, wherein the peroxidase is a non-animal haem peroxidase from class II (fungi) or class III (plants and algae).

3. A hair colour composition according to claim 2, wherein the peroxidase is obtained from the group consisting of Arabidopsis thaliana, horse radish, barley, peanut soybean, tobacco, and turnip (plants), Chlorophyta spirogyra (green algae), Arthromyces ramosus and Corprinus cinereus (fungi).

4. A hair colour composition according to claim 1, wherein the laccase originates from a fungal or a plant source such as those from the group consisting of the Aspergillus, Botrytis, Ceriporiopsis, Cerrena, Chaetomium, Coprinus, Coriolus, Neurospora, Panus, Phanerochaete, Pleurotus, Polyporus, Pycnoporus and Trametes genera, and Rhus vernicifera.

5. A hair colour composition according to claim 1, wherein the hair colour composition comprises 0.001-10, preferably 0.1-5% w/w (+)-gossypetin.

6. A hair colour composition according to claim 1, wherein the hair colour composition comprises 0.0001-3 preferably 0.001-1, most preferably 0.01-1% w/w hydrogen peroxide.

7. A hair colour composition according to claim 1, wherein the hair colour composition comprises 0.0001-5, preferably 0.0001-1% w/w peroxidase or laccase.

8. A hair colour composition according to claim 1, wherein the hydrogen peroxide generator comprises a hydrogen peroxide generating oxidase, a substrate and oxygen.

9. A hair colour composition according to claim 8, wherein the hydrogen peroxide generating oxidase is selected from the group consisting of (S)-2-hydroxy acid oxidase, D-galactose oxidase, glucose oxidase, coniferyl alcohol oxidase, glycolate oxidase, hexose oxidase, oxalate oxidase, amino acid oxidase and L-galactonolactone oxidase and the respective substrate is selected from the group consisting of (S)-2-hydroxy acid, D-galactose, glucose, coniferyl alcohol, α-hydroxy acids, D-glucose, oxalic acid, amino acid and L-galactono-1,4-lactone.

10. A hair colour composition according to claim 1, wherein the hydrogen peroxide generator is selected from the group consisting of (S)-2-hydroxy acid with (S)-2-hydroxy acid oxidase, D-galactose with D-galactose oxidase, glucose with glucose oxidase, coniferyl alcohol with coniferyl alcohol oxidase, α-hydroxy acids with glycolate oxidase, D-glucose with hexose oxidase, oxalic acid with oxalate oxidase, and L-galactono-1,4-lactone with L-galactonolactone oxidase, amino acid oxidase with amino acids, all in the presence of oxygen.

11. A hair colour composition according to claim 8, wherein the hair colour composition comprises 0.0001-5, preferably 0.001-1% w/w hydrogen peroxide generating oxidase.

12. A hair colour composition according to claim 11, wherein the hair colour composition comprises 0.01-10 preferably 0.1-5% w/w substrate.

13. A hair colour composition according to claim 1 additionally comprising a metal ion suitable for coordinating to gossypetin, or the product of the reaction of gossypetin in the presence of the peroxidase and hydrogen peroxide, or the laccase.

14. A hair colour composition according to claim 13, wherein the metal ion is selected from the group consisting of iron (II), iron (III), copper (I), copper (II), copper (III) and aluminium (III).

15. A hair colour composition according to claim 13, wherein the hair colour composition comprises 0.0001-2, preferably 0.001-0.1% w/w metal ion.

16. A method of colouring hair fibres, the method comprising the step of applying a hair colour composition according to claim 1 to hair fibres.
